(19) United States
(12) Patent Application Publication
(10) Pub. No.: US 2018/0326384 A1
(43) Pub. Date: Nov. 15, 2018

(54) MICROBUBBLES AND THEIR GENERATION

(71) Applicant: UCL Business PLC, London (GB)

(72) Inventors: Anjana KOTHANDARAMAN, London (GB); Mohan EDIRISINGHE, London (GB); Yiannis VENTIKOS, London (GB)

(21) Appl. No.: 15/774,712

(22) PCT Filed: Nov. 9, 2016
(86) PCT No.: PCT/GB2016/053497

§ 371 Date: May 9, 2018

(30) Foreign Application Priority Data
Nov. 10, 2015 (GB) 1519853.4

(51) Int. Cl. B01J 13/04; A61K 9/50

(52) U.S. Cl. CPC B01J 13/04; A61K 9/50

(57) ABSTRACT

Herein is described a process for producing microbubbles, the process comprising passing a first fluid medium through a first capillary, passing second fluid medium through a second capillary, such that the first fluid medium and second fluid medium meet at a junction and form microbubbles having a core of the first fluid medium and a shell of the second fluid medium, the microbubbles then flowing along an exit capillary, wherein a DC voltage and a superimposed AC voltage are applied to the exit capillary and microbubbles then exit the exit capillary.

### BACKGROUND

Gas-encapsulating microbubbles have gained substantial interest amongst researchers over the past two decades, branching out into various industries such as cosmetics, food engineering and in the biomedical field as diagnostic and therapeutic vehicles. Microbubbles play an important role in the preparation of contrast agents for ultrasound imaging. This is due to the useful properties they possess such as their high compressibility and ability to undergo linear and non-linear oscillations as an acoustic response to an ultrasound signal. As a result of an acoustic mismatch between soft tissue and biological fluids, microbubbles enable the resultant scattered signal to distinguish between the aforementioned components thus enhancing the imaging of tissue perfusion. The application of microbubbles has extended beyond contrast agents; their potential in drug delivery has been investigated across biological barriers such as the blood-brain barrier (BBB) assisted by focused ultrasound. The rhythmic oscillations from the applied ultrasound alters the vasculature of the endothelial wall, thus enhancing the over permeability of the targeted region which in turn improves overall drug delivery process.

Over the years, various methods have been used for the preparation of microbubbles. Mechanical agitation and sonication are two of the most extensively reported methods in literature. More recently co-axial electrohydrodynamic atomisation (CEHDA) was used to prepare multi-layered gas-bubbles. Wide bubble size distributions were recorded in methods such as sonication and mechanical agitation, which would require additional fractioning techniques to eradicate larger bubbles and retain monodispersity which is can be an important factor in biomedical applications. Although CEHDA displays a narrower size distribution in comparison to aforementioned techniques, but it did not reach monodispersity.

A technique for producing microbubbles with reasonable narrow size distributions involves using a capillary embedded T-junction. While this has been successful to an extent, there is a challenge to reduce the size of the bubbles, while maintaining a narrow size distribution and the integrity of the bubbles.

### SUMMARY OF THE INVENTION

In a first aspect, there is provided a process for producing microbubbles, the process comprising
passing a first fluid medium through a first capillary,
passing second fluid medium through a second capillary,
such that the first fluid medium and second fluid medium meet at a junction and form microbubbles having a core of the first fluid medium and a shell of the second fluid medium, the microbubbles then flowing along an exit capillary,
wherein a DC voltage and a superimposed AC voltage are applied to the exit capillary and microbubbles then exit the exit capillary.

In a second aspect, there is provided a device for producing microbubbles comprising
a first capillary,
a second capillary,
a junction, at which the first capillary and second capillary meet, and an exit capillary extending away from the junction,
a power supply connected to the exit-capillary, the power supply adapted to apply a DC voltage and a superimposed AC voltage to the exit capillary.

Producing microbubbles having or approaching monodispersity is a challenge. If a collection of microbubbles is monodisperse, in the present context this indicates that they have the same or approximately the same diameter as one another, i.e. a very narrow size distribution. As indicated above, it is also a challenge to reduce the size of microbubbles, while still maintaining their integrity and a narrow size distribution. It has been found that examples of the process and device described herein allow microbubbles to be produced of relatively small size, with favourable narrow size distribution, and without an apparent negative effect on their integrity. The technique avoids having to reduce capillary size to reduce microbubble diameter, which can be problematic in certain circumstances, since very small capillaries can become blocked relatively easily with viscous solutions.

### BRIEF DESCRIPTION OF THE DRAWINGS

FIG. 1A illustrated schematically an example of an apparatus for producing microbubbles as described herein.

FIG. 1B illustrates schematically an example of a gas-supply capillary and a liquid-supply capillary, which meet at a junction within a clear block of plastic and an exit capillary extending away from the junction.

FIGS. 2(a) to 2(f) show optical micrographs of microbubbles obtained by progressively increasing the applied DC voltage.

FIG. 3 shows a graph showing the effect on microbubble diameter of increasing the applied DC voltage at different AC peak-to-peak voltages.

FIG. 4 shows a graph showing the effect of microbubble diameter of increasing the applied DC voltage at different AC frequencies.

FIG. 5 shows a graph showing the effect of increasing the AC frequency on microbubble diameter.

### DETAILED DESCRIPTION

A microbubble in the present context may be a particle comprising a gas-containing or volatile-liquid-containing core and a liquid or solid outer shell. The size of a microbubble may be defined to be of the micron scale, e.g. having a diameter of less than 1 mm, optionally 500 μm or less, optionally 400 μm or less, optionally 300 μm or less, optionally 200 μm or less, optionally 150 μm or less, optionally 125 μm or less, optionally 100 μm or less, optionally 80 μm or less, optionally 50 μm or less, optionally 40 μm or less, optionally 30 μm or less, optionally 25 μm or less, optionally 20 μm or less, optionally 15 μm or less, optionally 10 μm or less. The diameter of a microbubble may be determined by a suitable microscope such as an optical microscope, e.g. by placing a microbubble, or a collection of microbubbles, on a glass slide, and, for a given microbubble, measuring the largest diameter across the microbubble. The gas-containing core may be a core that, at a temperature of 37° C., i.e. the temperature of a human body, contains a gas. Below 37° C., the core may contain a gas or a volatile liquid that forms a gas as the temperature is raised to 37° C.

### First and Second Capillaries

The first and second capillaries may have internal diameters that are the same as or different from one another. An internal diameter is the diameter across the channel in a capillary through which a fluid can flow. The first and second capillaries may have internal diameters of at least 10 μm, optionally at least 50 μm, optionally at least 75 μm, optionally at least 100 μm, optionally at least 150 μm. The first and second capillaries may have internal diameters of 300 μm or less, optionally at 200 μm or less, optionally 150 μm or less, optionally 100 μm or less. The first and second capillaries may have internal diameters of from 10 μm to 300 μm, optionally from 50 μm to 200 μm, optionally from 50 μm to 150 μm, optionally from 75 μm to 125 μm, optionally from 50 μm to 100 μm, optionally from 100 μm to 150 μm.

The first and second capillaries preferably comprise a non-electrically conducting material. The first and second capillaries may comprise a plastic. First and second capillaries may comprise a fluorinated hydrocarbon plastic, such as fluorinated ethylene (e.g. polytetrafluoroethylene, PTFE) or fluorinated propylene, or a co-polymer of fluorinated ethylene and fluorinated propylene (sometimes termed fluorinated ethylene propylene, of FEP).

The first fluid medium may be supplied at a constant rate through the first capillary. The second fluid medium may be supplied at a constant rate through the second capillary. Both first and second fluid mediums may be passed through first and second capillaries at a constant rate, and the rates may be the same as or different to one another.

The first fluid medium may be supplied at a rate of from 1 μl/min to 2000 μl/min, optionally from 25 μl/min to 1000 μl/min, optionally from 25 to 500 μl/min, optionally from 25 μl/min to 300 μl/min, optionally from 25 μl/min to 300 μl/min, optionally from 25 μl/min to 200 μl/min, optionally from 50 μl/min to 150 μl/min, optionally from 75 μl/min to 125 μl/min.

The second fluid medium may be supplied at a rate of from 1 μl/min to 2000 μl/min, optionally from 25 μl/min to 1000 μl/min, optionally from 25 to 500 μl/min, optionally from 25 μl/min to 300 μl/min, optionally from 25 μl/min to 300 μl/min, optionally from 25 μl/min to 200 μl/min, optionally from 50 μl/min to 150 μl/min, optionally from 75 μl/min to 125 μl/min.

The second capillary may disposed at an angle of from 0° to 90° from the first capillary. The second capillary may be disposed at perpendicular or substantially perpendicular (e.g. within 5° from perpendicular) from the first capillary.

The first and second fluid mediums may be supplied under pressure. The pressure may be from 100 to 500 kPa, in some examples from 150 to 350 kPa.

### Exit Capillary

The exit capillary may have an internal diameter that is the same as or different from the first capillary or the second capillary. The exit capillary may have an internal diameter that is the same as or substantially the same as (e.g. within 10 μm of) the first and second capillaries. The exit capillary may have an internal diameter of at least 10 μm, optionally at least 50 μm, optionally at least 75 μm, optionally at least 100 μm, optionally at least 150 μm. The exit capillary may have an internal diameter of 300 μm or less, optionally at 200 μm or less, optionally 150 μm or less, optionally 100 μm or less. The exit capillary may have an internal diameter of from 10 μm to 300 μm, optionally from 50 μm to 200 μm, optionally from 50 μm to 150 μm, optionally from 75 μm to 125 μm, optionally from 50 μm to 100 μm, optionally from 100 μm to 150 μm.

The exit capillary may be disposed so that it is substantially co-axial (i.e. in line with) the first capillary, and, optionally, the second capillary may be disposed at an angle of from 0° to 90° from the first capillary. The exit capillary may be disposed so that it is substantially co-axial (i.e. in line with) the first capillary, and, optionally, the second capillary may be disposed at perpendicular or substantially perpendicular (e.g. within 5° from perpendicular) from the first capillary. A T-junction may be formed from the arrangement of the first, second and exit capillaries, with the exit capillary being perpendicular or substantially perpendicular to the first and second capillaries.

The exit capillary preferably comprises an electrically conducting material. The exit capillary preferably comprises a metal. The metal may be selected from, for example, an elemental metal or a metal alloy. The metal may, for example, comprise steel.

The junction may be formed by a cavity in a body in which the first, second and exit capillaries are disposed. Each of the first, second and exit capillaries may have a portion that is contained within the body, and the remaining portion may extend out of the body, and, for example, for the first and second capillaries, be connected to a supply of, respectively, the first fluid medium and second fluid medium. The body may be a plastic or glass body. The ends of the first and exit capillary may be separated by a distance that is the same or less than the internal diameter of the first and/or exit capillary. The ends of the first and exit capillary may be separated by a distance of 200 μm or less, optionally, 150 μm or less, optionally 100 μm or less, optionally 50 μm or less.

### Applying a Voltage to the Exit Capillary

A DC voltage and a superimposed AC voltage are applied to the exit capillary. Where a value of DC or AC voltage is referred to herein, this is the voltage applied relative to ground voltage, at which the microbubbles are collected in a receptacle or on a slide after they exit the exit capillary.

The DC voltage applied to the exit capillary may be from 1 kV to 12 kV, optionally from 2 kV to 10 kV. The DC voltage applied to the exit capillary may be from 4 kV to 8 kV, in some examples from 5 kV to 7 kV, in some examples about 6 kV.

When an AC voltage is superimposed on the DC voltage, the AC voltage will have a peak-to-peak voltage. A peak-to-peak voltage is the difference between the maximum and minimum of the waveform of the AC oscillation. When a DC voltage is applied and an AC voltage superimposed, the AC voltage will oscillate about the DC voltage, with the difference between the peaks and troughs in the AC waveform being the peak-to-peak voltage. As an example, if a DC voltage of V₁ is applied, and AC voltage superimposed with a peak-to-peak voltage V₂, the voltage of the exit capillary will oscillate from V₁−½V₂ to V₁+½V₂. As an example, if a DC voltage of 6 kV is applied to the exit capillary and an AC voltage is applied with a peak-to-peak voltage of 2 kV, the voltage of the exit capillary will oscillate from 5 kV to 7 kV.

In an embodiment, a peak-to-peak AC voltage of 3 kV or less is applied to the exit capillary. Optionally, a peak-to-peak AC voltage of from 1.5 kV to 2.5 kV, optionally about 2 kV, is applied to the exit capillary. Keeping the peak-to-peak AC voltage below 3 kV has been found to improve the size reduction effects of the AC voltage, particularly at high frequencies.

The frequency of the superimposed AC voltage applied to the exit capillary may be at least 50 Hz, optionally at least 100 Hz, optionally at least 300 Hz, optionally at least 300 Hz, optionally at least 800 Hz, optionally at least 2000 Hz, optionally at least 5000 Hz, optionally at least 10,000 Hz, optionally at least 1 MHz, optionally at least 5 MHz, optionally at least 8 MHz. Increasing the AC frequency has been found to decrease the size of the microbubbles produced. Optionally, the frequency is from 800 Hz to 15 MHz, optionally from 1000 Hz to 15 MHz, optionally from 2000 Hz to 15 MHz, optionally from 2000 Hz to 12 MHz, optionally from 2000 Hz to 10 MHz, optionally from 5000 Hz to 15 MHz, optionally from 10,000 Hz to 15 MHz, optionally from 1 MHz to 15 MHz, optionally from 5 MHz to 15 MHz, optionally from 1 MHz to 10 MHz. The above-mentioned frequencies are preferably applied with a peak-to-peak AC voltage of 3 kV or less, preferably from 1.5 kV to 2.5 kV, e.g. 2 KV, and DC voltage of from 3 kV to 9 kV, preferably 4 kV to 8 kV. This has been found to decrease microbubble size, yet produce microbubbles with a small size distribution, which can approach monodispersity.

The waveform of the AC voltage may be sinusoidal, square wave or triangular.

### First Fluid Medium

The first fluid medium is supplied through the first capillary. The first fluid medium is preferably supplied at a constant rate.

The first fluid medium may be any suitable medium for forming a core for microbubbles, which may be a gas-containing core, when the microbubbles are at 37° C. The first fluid medium, on being passed through the first capillary may be a gas or a volatile fluid for forming a gas. A volatile fluid for forming a gas may be a fluid having a boiling point from 20° C.-50° C., e.g. from 20° C.-37° C. at standard pressure (100,000 Pa). The first fluid medium may comprise a species selected from nitrogen, oxygen, air, carbon dioxide, hydrogen, nitrous oxide, helium, argon, xenon, krypton, nitric oxide, sulphur fluorides (e.g. sulphur hexafluoride or disulphur decafluoride), a fluorocarbon and a hydrocarbon. A fluorocarbon includes partially fluorinated carbon-containing compounds. The fluorocarbon may be a perfluorocarbon (a fully fluorinated carbon-containing compound). The fluorocarbon may be selected from tetrafluoromethane, hexafluoroethane, octafluoropropane, decafluorobutane, or perfluoro-isobutan, perfluorohexane, perfluoropentane, perfluorocyclopentane, 1,1,2-trichlorotrifluoroethane and sulfur hexafluoride. The hydrocarbon may be a C1-C10, optionally a C1-C5 hydrocarbon, which may be a C1-C5 alkane such as methane, ethane, propane, butane and pentane. The hydrocarbon may be straight-chain or branched. The hydrocarbon may be an alkane, an alkene (e.g. ethylene or propene) or an alkyne (e.g. acetylene or propyne). The hydrocarbon may be selected from methane, ethane, propane, butane, pentane, cyclopentane, methylene chloride, pentane and hexane. As will be appreciated, other gases may be used, which may be more or less volatile than the gases specifically mentioned herein.

The first fluid medium may be selected from hexafluoro acetone, isopropyl acetylene, allene, tetrafluoro-allene, boron trifluoride, isobutane, 1,2-butadiene, 2,3-butadiene, 1,3-butadiene, 1,2,3-trichloro-2-fluoro-1,3-butadiene, 2-methyl-1,3-butadiene, hexafluoro-1,3-butadiene, butadiyne, 1-fluoro-butane, 2-methyl-butane, decafluorobutane, 1-butene, 2-butene, 2-methyl-1-butene, 3-methyl-1-butene, perfluoro-1-butene, perfluoro-2-butene, 4-phenyl-3-butene-2-one, 2-methyl-1-butene-3-yne, butyl nitrate, 1-butyne, 2-butyne, 2-chloro-1,1,1,4,4,4-hexafluorobutyne, 3-methyl-1-butyne, perfluoro-2-butyne, 2-bromobutyraldehyde, carbonyl sulfide, crotononitrile, cyclobutane, methyl-cyclobutane, octafluoro-cyclobutane, perfluorocyclobutene, 3-chlorocyclopentene, octafluorocyclopentene, cyclopropane, 1,2-dimethyl-cyclopropane, 1,1-dimethylcyclopropane, 1,2-dimethyl-cyclopropane, ethylcyclopropane, methylcyclopropane, 3-ethyl-3-methyl diaziridine, 1,1,1-trifluorodiazoethane, dimethyl amine, hexafluorodimethylamine, dimethylethylamine, bis(dimethylphosphine)amine, perfluorohexane, 2,3-dimethyl-2-norbornane, perfluorodimethylamine, dimethyloxonium chloride, 1,3-dioxolane-2-one, 4-methyl-1,1,1,2-tetrafluoroethane, 1,1,1-trifluoroethane, 1,1,2,2-tetrafluoroethane, 1,1,2-trichloro-1,2,2-trifluoroethane, 1,1-dichloroethane, 1,1-dichloro-1,2,2,2-tetrafluoroethane, 1,2-difluoroethane, 1-chloro-1,1,2,2,2-pentafluoroethane, 2-chloro-1,1-difluoroethane, 1,1-dichloro-2-fluoroethane, 1-chloro-1,1,2,2-tetrafluoroethane, 2-chloro-1,1-difluoroethane, chloroethane, chloropentafluoroethane, dichlorotrifluoroethane, fluoroethane, hexafluoroethane, nitropentafluoroethane, nitrosopentafluoroethane, perfluoroethylamine, ethyl vinyl ether, 1,1-dichloroethane, 1,1-dichloro-1,2-difluoroethane, 1,2-difluoroethane, methane, trifluoromethanesulfonylchioride, trifluoromethanesulfonylfluoride, bromodifluoronitrosomethane, bromofluoromethane, bromochlorofluoromethane, bromotrifluoromethane, chlorodifluoronitromethane, chlorodinitromethane, chlorofluoromethane, chlorotrifluoromethane, chlorodifluoromethane, dibromodifluoromethane, dichlorodifluoromethane, dichlorofluoromethane, difluoromethane, difluoroiodomethane, disilanomethane, fluoromethane, iodomethane, iodotrifluoromethane, nitrotrifluoromethane, nitrosotrifluoromethane, tetrafluoromethane, trichlorofluoromethane, trifluoromethane, 2-methylbutane, methyl ether, methyl isopropyl ether, methyllactate, methylnitrite, methylsulfide, methyl vinyl ether, neon, neopentane, nitrogen (N₂), nitrous oxide, 1,2,3-nonadecane-tricarboxylic acid-2-hydroxytrimethylester, 1-nonene-3-yne, oxygen (O₂), 1,4-pentadiene, n-pentane, perfluoropentane, 4-amino-4-methylpentan-2-one, 1-pentene, 2-pentene (cis), 2-pentene (trans), 3-bromopent-1-ene, perfluoropent-1-ene, tetrachlorophthalic acid, 2,3,6-trimethylpiperidine, propane,1,1,1, 2,2,3-hexafluoropropane, 1,2-epoxypropane, 2,2-difluoropropane, 2-aminopropane, 2-chloropropane, heptafluoro-1-nitropropane, heptafluoro-1-nitrosopropane, perfluoropropane, propene, hexafluoropropane, 1,1,1,2,3,3-hexafluoro-2,3 dichloropropane, 1-chloropropane, chloropropane-(trans), 2-chloropropane, 3-fluoropropane, propyne, 3,3,3-trifluoropropyne, 3-fluorostyrene, sulfur hexafluoride, sulfur (di)-decafluoride(S₂F₁₀), 2,4-diaminotoluene, trifluoroacetonitrile, trifluoromethyl peroxide, trifluoromethyl sulfide, tungsten hexafluoride, vinyl acetylene, vinyl ether, and xenon.

### Second Fluid Medium

The second fluid medium may contain any suitable component for forming a shell of a microbubble. The second fluid medium may have a boiling point, measured at standard pressure (100,000 Pa) that is above the boiling point of the first fluid medium. The boiling point of the second fluid medium may be 80° C. or above, optionally 100° C. or above, optionally 200° C. or above, optionally 300° C. or above.

The second fluid medium may comprise or consist of a carrier liquid. The carrier liquid may comprise a solvent, such as water and/or an organic solvent. The organic solvent may comprise a non-polar solvent and/or a polar solvent. The organic solvent may comprise an aprotic solvent and/or a protic solvent. Non-polar solvents include, but are not limited to, pentane, cyclopentane, hexane, benzene, toluene, 1,4-dioxane, chloroform, and diethylether. The solvent may comprise a polar aprotic solvent, optionally selected from dichloromethane, tetrahydrofuran, ethylacetate, acetone, dimethylformamide, acetonitrile, dimethyl sulphoxide. The solvent may comprise a polar protic solvent, optionally selected from water, formic acid, n-butanol, isopropanol, n-propanol, ethanol, methanol and acetic acid. The solvent may comprise a polyol, such as a C2 to C5 polyol, such as glycerol. The organic solvent may comprise a hydrocarbon. The hydrocarbon may comprise an aromatic or an aliphatic hydrocarbon. The hydrocarbons may be selected from, but are not limited to, pentane, cyclopentane, hexane, cyclohexane and benzene.

The second fluid medium may comprise a surfactant or a polymer. The surfactant or polymer may be dissolved or suspended in a carrier liquid, which may be as described herein. The surfactant may include a lipid, such as a phospholipid. The lipid may comprise a lecithin (i.e. a phosphatidylcholine). The lecithin may be a natural or a synthetic lecithin. The lipid may be a natural lecithin selected from egg yolk lecithin and soya bean lecithin. The lipid may be a synthetic lecithins selected from dimyristoylphosphatidylcholine, dipalmitoylphosphatidylcholine or distearoylphosphatidylcholine; phosphatidic acids; phosphatidylethanolamines; phosphatidylserines; phosphatidylglycerols; and phosphatidylinositols.

The second fluid medium preferably comprises a biocompatible substance. The second fluid medium may comprise a substance selected from poly(lactic-co-glycolic acid) (PLGA), polycaprolactone (PCL), polymethylsilsesquioxane (PMSQ), perfluorohexane (PFH), perfluoropentane (PFP), polyurethane, starch, albumin, polyethylene oxide (PEO), glycerol and an oil, such as olive oil. The albumin may be selected from bovine serum albumin and human serum albumin.

Particularly preferred polymers include, but are not limited to, sodium polystyrene sulfonate (PSS), polyethers, such as a polyethylene oxide (PEO), polyoxyethylene glycol or polyethylene glycol (PEG), polyethylene imine (PEI), a biodegradable polymer such as a polylactic acid, polycaprolactone, polyglycolic acid, poly(lactic-co-glycolic acid) (PLGA), polymethylsilsesquioxane (PMSQ) and copolymers, derivatives, and mixtures thereof. Other polymers that may be used include those well known to those of skill in the art to be used in cell cultures, implants, regenerative, therapeutic, and pharmaceutical compositions. One such example is polyvinylpyrrolidone (PVP).

Optionally, the polymer may have a property selected from: being positively-charged being (cationic), being negatively-charged (anionic), being polyethylene glycol(PEG)-ylated, being covered with a zwitterion, being hydrophobic, being superhydrophobic (for example having with water contact angles in excess of 150°), being hydrophilic, being superhydrophilic (for example, where the water contact angle is near or at 0°), being olephobic/lipophobic, being olephilic/lipophilic, and/or nanostructured, among others.

The polymer may be a water-soluble and/or hydrophilic polymer, which may be selected from biocompatible polymers, including, but not limited to, cellulose ether polymers, including those selected from the group consisting of hydroxyl alkyl cellulose, including hydroxypropyl methyl cellulose (HPMC), hydroxypropyl cellulose (HPC), hydroxyethyl cellulose (HEC), methyl cellulose (MC), carboxymethyl cellulose (CMC), and mixtures thereof.

The polymer may also be selected from polyvinylpyrrolidone, vinyl acetate, polyvinylpyrrolidone-vinyl acetate copolymers, polyvinyl alcohol (PVA), acrylates and polyacrylic acid (PAA), including polyacrylate polymer, vinylcaprolactam/sodium acrylate polymers, methacrylates, poly(acryl amide-co-acrylic acid) (PAAm-co-AA), vinyl acetate and crotonic acid copolymers, polyacrylamide, polyethylene phosphonate, polybutene phosphonate, polystyrene, polyvinylphosphonates, polyalkylenes, and carboxy vinyl polymer.

The polymer may be selected from water insoluble or hydrophobic polymers including, but not limited to, cellulose acetate, cellulose nitrate, ethylene-vinyl acetate copolymers, vinyl acetate homopolymer, ethyl cellulose, butyl cellulose, isopropyl cellulose, shellac, hydrophobic silicone polymer (e.g., dimethylsilicone), polymethyl methacrylate (PMMA), cellulose acetate phthalate and natural or synthetic rubber; siloxanes, such as polydimethylsiloxane (PDMS), cellulose, polyethylene, polypropylene, polyesters, polyurethane and nylon, including copolymers, derivatives, and combinations thereof.

The polymers may be crosslinked, optionally after formation of the microbubbles, for example by the application of heat, ionizing radiation or other methods of curing and treating polymers known to those of skill in the art.

Optionally, the polymer may be selected from sodium alginate, carrageenan, xanthan gum, gum acacia, Arabic gum, guar gum, pullulan, agar, chitin, chitosan, pectin, karaya gum, locust bean gum, various polysaccharides; starches such as maltodextrin, amylose, corn starch, potato starch, rice starch, tapioca starch, pea starch, sweet potato starch, barley starch, wheat starch, modified starch (e.g., hydroxypropylated high amylose starch), dextrin, levan, elsinan and gluten; and proteins such as collagen, whey protein isolate, casein, milk protein, soy protein, keratin, and gelatin.

As indicated, the surfactant or polymer may be dissolved or suspended in a carrier liquid, which may be as described herein. The carrier liquid of the first fluid medium may be any suitable liquid in which the surfactant or polymer can be dissolved and/or suspended. The surfactant or polymer may be completely dissolved in the carrier liquid.

The second fluid medium preferably has a dynamic viscosity of 1 mPa·s or more, optionally a dynamic viscosity of 2 mPa·s or more, optionally a dynamic viscosity of 3 mPa·s or more, optionally a dynamic viscosity of 5 mPa·s or more, optionally a dynamic viscosity of 7 mPa·s or more, optionally a dynamic viscosity of 8 mPa·s or more, optionally a dynamic viscosity of 10 mPa·s or more, optionally a dynamic viscosity of 20 mPa·s or more, optionally a dynamic viscosity of 30 mPa·s or more, optionally a dynamic viscosity of 50 mPa·s or more, optionally a dynamic viscosity of 80 mPa·s or more, optionally a dynamic viscosity of 50 mPa·s or more, optionally a dynamic viscosity of 100 mPa·s or more. The dynamic viscosity is measured at standard temperature (25° C.) and pressure (101.325 kPa). Dynamic viscosity values can be measured according to a standard method known to those skilled in the art, for example by using a U-tube viscometer (sometimes termed an Ostwald viscometer) such as a commercially available Schott Instruments GmbH, Mainz, Germany, or a rotational viscometer. Ethanol may used as a calibrating medium in the relevant measurement equipment, if necessary.

The second fluid medium preferably has a surface tension of 20 mNm⁻¹ or more, optionally 25 mNm⁻¹ or more, optionally 30 mNm⁻¹ or more, optionally 40 mNm⁻¹ or more, optionally 40 mNm⁻¹ or more. The second fluid medium preferably has a surface tension of from 20 mNm⁻¹ to 70 mNm⁻¹, optionally from 30 mNm⁻¹ to 70 mNm⁻¹, optionally from 40 mNm⁻¹ to 60 mNm⁻¹, optionally from 50 to 60 mNm⁻¹. The surface tension of the second fluid medium is measured at standard temperature (25° C.) and pressure (100,000 Pa). The surface tension of the second fluid medium is preferably more than the surface tension of the first fluid medium, when measured under the same conditions. Surface tension can be measured according to a standard method known to those skilled in the art, for example by using a tensiometer, e.g. a commercially available Wilhelmy Plate tensiometer (Kruss, Hamburg, Germany). Ethanol may used as a calibrating medium in the relevant measurement equipment, if necessary.

The conductivity of the second fluid medium may be at least 0.1 μSm⁻¹, optionally at least 1 μSm⁻¹, optionally at least 10 μSm⁻¹, optionally at least 100 μSm⁻¹, optionally at least 1000 μSm⁻¹. The conductivity of the second fluid medium may be from 0.1 to 10 μSm⁻¹, optionally from 0.5 to 5 μSm⁻¹. Conductivity in this context refers to the electrical conductivity. The conductivity of the second fluid medium is measured at standard temperature (25° C.) and pressure (100,000 Pa). Conductivity can be measured according to a standard method known to those skilled in the art, for example by using a conductivity probe, such as the commercially available Jenway 3540 conductivity meter, available from Bibby Scientific, Stone, UK. Ethanol may used as a calibrating medium in the relevant measurement equipment, if necessary.

The microbubbles may comprise an active agent, for example in the core region and/or the shell. To produce a microbubble containing an active agent in the core, an active agent may be included in the first fluid medium. To produce a microbubble containing an active in the shell, an active agent may be included in the second fluid medium.

The active agent may be or comprise a diagnostic agent. The diagnostic agent may be an agent suitable for use in a technique selected from, but not limited to, diagnostic medical imaging procedures (for example, radiographic imaging (x-ray), fluorescence spectroscopy, Forster/fluorescent resonance energy-transfer (FRET), computed tomography (CT scan), magnetic resonance imaging (MRI), positron emission tomography (PET), other nuclear imaging, and the like. The diagnostic agent may be an agent for use in diagnostic imaging, for example a contrast agents, such as barium sulfate for use with MRI, for example, or fluorescein isothiocyanate (FITC).

The active agent may be a therapeutic agent. The therapeutic agent may be a drug for the treatment or prevention of a disease.

The active agent may be selected from pharmaceutical and/or cosmetic active agents, which may be selected from growth factors; growth factor receptors; transcriptional activators; translational promoters; antiproliferative agents; growth hormones; anti-rejection drugs; anti-thrombotic agents; anti-coagulants; stem cell or gene therapies; antioxidants; free radical scavengers; nutrients; co-enzymes; ligands; cell adhesion peptides; peptides; proteins; nucleic acids; DNA; RNA; sugars; saccharides; nutrients; hormones; antibodies; immunomodulating agents; growth factor inhibitors; growth factor receptor antagonists; transcriptional repressors; translational repressors; replication inhibitors; inhibitory antibodies; cytotoxin; hormonal agonists; hormonal antagonists; inhibitors of hormone biosynthesis and processing; antigestagens; antiandrogens; anti-inflammatory agents; non-steroidal anti-inflammatory agents (NSAIDs); analgesics; COX-I and II inhibitors; antimicrobial agents; antiviral agents; antifungal agents; antibiotics; anti-proliferative agents; antineoplastic/antiproliferative/anti-miotic agents; anesthetic, analgesic or pain-killing agents; antipyretic agents, prostaglandin inhibitors; platelet inhibitors; DNA de-methylating agents; cholesterol-lowering agents; vasodilating agents; endogenous vasoactive interference agents; angiogenic substances; cardiac failure active ingredients; polysaccharides; sugars; targeting toxin agents; aptamers; quantum dots; nano-materials; nano-crystals; and combinations thereof.

At least some of the microbubbles produced may have a diameter of 200 μm or less, optionally 150 μm or less, optionally 125 μm or less, optionally 100 μm or less, optionally 80 μm or less, optionally 50 μm or less, optionally 40 μm or less, optionally 30 μm or less, optionally 25 μm or less, optionally 20 μm or less, optionally 15 μm or less, optionally 10 μm or less.

At least some of the microbubbles produced may have a diameter of from 0.5 μm to 200 μm, optionally from 0.5 μm to 150 μm, optionally from 0.5 μm to 100 μm, optionally from 0.5 μm to 50 μm, optionally from 0.5 μm to 30 μm, optionally from 0.5 μm to 20 μm, optionally from 0.5 μm to 10 μm, optionally from 1 μm to 10 μm. “At least some of the microbubbles” may indicate ‘at least 90% by number of the microbubbles’, in some examples ‘at least 95% by number of the bubbles’.

The microbubbles produced may be substantially monodispersed microbubbles. Monodispersed microbubbles may have a size distribution in which all the microbubbles produced have a diameter that is within 10% (optionally within 5%, optionally within 2%) of an number average diameter, which may be within the ranges of diameters given above.

The first fluid medium may comprises or consists of a volatile liquid. In an embodiment, the volatile liquid is a liquid that has a boiling point not higher than 50° C. above the temperature of the environment into which the microbubbles pass into when exiting the exit capillary, optionally not higher than 40° C. above the temperature of the environment into which the microbubbles pass into when exiting the exit capillary, optionally not higher than 35° C. above the temperature of the environment into which the microbubbles pass into when exiting the exit capillary, optionally not higher than 30° C. above the temperature of the environment into which the microbubbles pass into when exiting the exit capillary. For example, if the temperature of the environment into which the microbubbles pass into when exiting the exit capillary is 25° C., preferably, the volatile liquid has a boiling point of 75° C. or less. The volatile liquid may have a boiling point of less than 100° C., optionally less than 80° C., optionally less than 70° C., optionally less than 60° C., optionally less than 50° C.

The temperature of the environment into which the microbubbles pass into when exiting the exit capilliary may be any suitable temperature. The process may be carried out such that the temperature of the environment into which the microbubbles pass into when exiting the exit capilliary is at or above the boiling point of first fluid medium. The temperature of the environment into which the microbubbles pass into when exiting the exit capillary may be above 15° C., optionally above 20° C., optionally above 25° C. The temperature of the environment into which the microbubbles pass into when exiting the exit capilliary may be less than 150° C., optionally less than 100° C., optionally less than 80° C., optionally less than 60° C., optionally less than 40° C. The temperature of the environment into which the microbubbles pass into when exiting the exit capillary may be from 10° C. to 40° C., optionally from 20° C. to 30° C.

The environment into which the microbubbles pass into when exiting the exit capillary may or may not contain a gas. Preferably, the environment into which the microbubbles pass into when exiting the exit capillary contains a gas, which may comprise a gas selected from nitrogen, oxygen, and a gas from Group 18 of the periodic table. The gas from Group 18 of the periodic table may be selected from helium, neon and argon. The environment into which the microbubbles pass into when exiting the exit capillary may contain air.

The environment into which the microbubbles pass into when exiting the exit capillary may contain a gas and be at a pressure of from 80 kPa to 120 kPa, optionally 90 to 110 kPa, optionally 95 to 105 kPa, optionally around standard pressure (100,000 Pa).

Herein is disclosed a device for producing microbubbles comprising
a first capillary
a second capillary
a junction, at which the first capillary and second capillary meet, and an exit capillary extending away from the junction,
a power supply connected to the exit-capillary, the power supply adapted to apply a DC voltage and a superimposed AC voltage to the exit capillary.

The first capillary may be fluidly connected to a supply of the first fluid medium. If the first fluid medium is a gas, the supply may be a suitable gas canister containing the gas, the flow of which can be controlled as desired. If the first fluid medium is a liquid, e.g. a volatile liquid or fluid as described herein, the supply of the first fluid medium may comprise or be a syringe pump.

The second capillary may be fluidly connected to a supply of the second fluid medium. The second fluid medium is typically a liquid. The supply of the second fluid medium may comprise or be a syringe pump.

A power supply may be connected to the exit-capillary and the power supply may be adapted to apply a DC voltage and, optionally, a superimposed AC voltage to the exit capillary. The power supply may comprise a voltage amplifier, e.g. a high voltage amplifier, which may be associated with a DC power supply unit and/or an AC waveform generator and, optionally, an oscilloscope, to check the waveform produced.

A ground electrode may be present, which may be at or near a receptacle to collect the microbubbles.

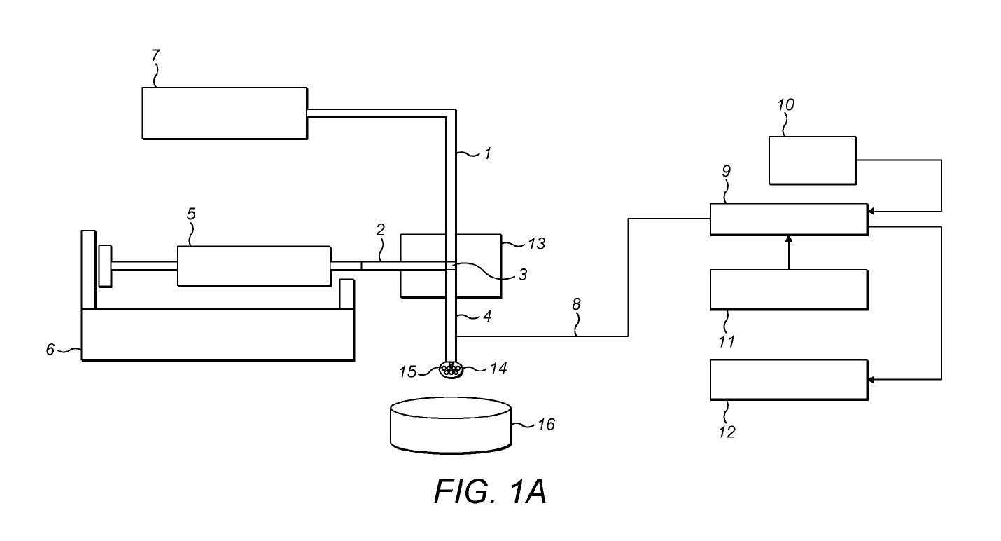

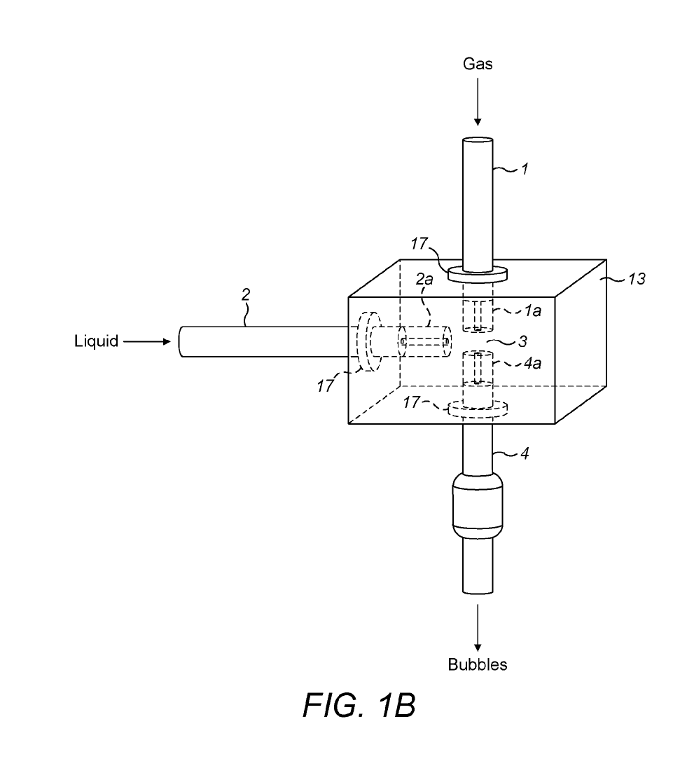

In FIG. 1A is shown, schematically, a first capillary (1), for supplying a first fluid medium from a first fluid supply (7), and a second capillary (2), for supplying a second fluid medium from a second fluid supply (5). The first fluid medium and the second fluid medium meet at junction (3) within a plastic (e.g. polydimethylsiloxane) or glass block (13). Microbubbles are formed and they then flow along an exit capillary (4). The exit capillary (4) is connected by an electrical cable (8) to a high voltage amplifier (9), which is in turn connected to a DC power supply unit (10), a waveform generator (11), to generate an AC waveform and an oscilloscope (12). The high voltage amplifier (9) amplifies the DC voltage from the DC power supply unit. A DC voltage with a superimposed AC voltage is applied to the exit capillary (8). Microbubbles (15) generally collect within a drop of liquid (14) at the open end of the exit capillary (4). They may be collected within a receptacle or on a slide (16).

FIG. 1B shows in more detail, again schematically, the first (1), second (2) and exit (4) capillaries within the block (13) and the junction (3). Each capillary extends through a collar (17) that screws into the block (13), to allow easy removal of the capillaries when desired. The end-sections of the capillaries (1a, 2a, and 4a) within the block (13) are shown see-through, for clarity.

### Examples

1. Materials and Methods

### Solution

Glycerol of 99% purity purchased from Sigma Aldrich, UK was diluted with distilled water. PEG-40-S (Polyethylene Glycol-40-Stereate, Sigma, Aldrich, UK) is a non-ionic/neutral surfactant. It was added into the solution to reduce surface tension and facilitate bubble formation. The physical properties of the solution used to prepare bubbles is shown in table 1.

TABLE 1

| | | Surface | Electrical | |
|---|---|---|---|---|
| | Viscosity | Tension | Conductivity | Density |
| Solution | (mPa s) | (mN m⁻¹) | (μS m⁻¹) | (kg m³) |
| 50% wt. Glycerol + | 8 | 54 | 2.0 | 1100 |
| 1% wt. PEG- 40-S | | | | |

### Characterisation of Solution

The density of the solutions was measured using a 25 ml DIN ISO 3507 Gay-Lussac type density bottle (VWR International, Lutterworth, UK). The viscosity was investigated using an Ostwald Viscometer (Schott Instruments GmbH, Mainz, Germany). The surface tension of the solution was measured using a Wilhelmy Plate tensiometer (Kruss, Hamburg, Germany) and the electrical conductivity was studied using a Jenway 3540 conductivity meter (Bibby Scientific, Stone, UK).

### Experimental Setup

The purpose of this experimental set-up (as shown schematically in FIG. 1A) is to superimpose an AC field onto a specified DC field.

A waveform generator (11) allows this by applying an offset to the output signal. This resultant signal ranging from 0-10V is used to drive the input of the programmable High Voltage Amplifier (9, HVA) (Ultravolt, Ronkonkoma, N.Y.) which amplifies 0-10V supplied by a 34V DC power supply unit (10) to 0-20 kV. The output signal can be viewed using an oscilloscope (12) by connecting it to the appropriate output pin. The amplifier (9) has a 15-pin D-type Female socket, which enables the complete programming of the unit.

The High Voltage amplifier (9, HVA) is used to amplify a low voltage input (10). This unit is fitted with a differential operational amplifier. Differential amplifiers generate an amplified output of the difference between two input voltage signals. The positive(+) or live terminal from the voltage source is connected to the positive (+) pin of the Voltage programming pin (V2) and negative(−) pin is connected to ground. Therefore, the output voltage can be expressed as:

Vₒᵤₜ=V₂−V₁

An alligator clip was soldered to the output cable (8) of the HVA (9) which was fastened securely to the stainless steel capillary (4). An ‘O’ connector was soldered to a wire, which screwed on to the High Voltage return and connected to the platform to complete the circuit. The parameters being tested were set on the Frequency Generator, and the low voltage power supply enabled the HVA to power-up. Prior to any experiments, the FEP capillaries (1,2) and the stainless steel capillary (4) were inspected for blockages, to ensure there was no obstruction to flow.

### Calibration of High Voltage Amplifier

In order to verify that the voltage being supplied by the HVA (9) is the same as the one being supplied to the load, a High Voltage Probe (Fluke,80k-40) embedded with 10MΩ resistor which acts as a 1000:1 potential divider. It was connected to a digital multimeter (Fluke, 10 A ac, 1000V AC). The high impedance HV probe essentially steps-down the measured voltage by a factor of 1000V, i.e. a measurement of 2 kV was recorded as 2V on the multimeter. The AC component was also checked, by changing the setting on the multimeter. It was recorded as an rms voltage, this was converted into a P-P voltage by:

$${V}_{rm s} = \frac{{V}_{0}}{√ 2}$$

### Calibration of Input and Output Signals

The signal that drives the inputs of the HVA was verified against the output signal from the HVA. A direct connection from the waveform generator was made to channel 1, using a BNC T-connector piece, from which the second connection was made to the input of the HVA (9). A direct connection was made from the monitoring pin on the HVA (9), to view the output signal. The signals were relatively in phase, apart from a slightly distorted output signal due to noise prevalent in the amplifier, which generates a slight harmonic distortion.

### Bubble Generation

Two Fluorinated Ethylene Propylene tubes (1,2), of 100 μm internal diameter were connected to the two inlets of the T-Junction (13) as shown schematically in FIG. 1B. A stainless steel capillary (4), of the same dimensions was connected to the outlet to provide a conductive surface to apply the electric field. The top inlet was connected to a pressurised gas tank comprised of nitrogen, and the second inlet was connected to a stainless steel syringe that supplies the solution at a constant flow rate. The fluid was injected at a constant flowrate of 100 μl/min, with a constant pressure of 256 kPa. These two fluids meet at the junction area, where formation of bubbles occur, which traverse through the outlet capillary and are collected on a glass slide. All experiments were conducted under an ambient temperature of 22° C. and a relative humidity of 36.6%.

### Characterisation of Bubbles

Microbubbles were collected on a glass slide and observed under an optical microscope (Nikon Co, Tokyo, Japan) immediately after generation. The optical micrographs were studied using ImageJ 1.48v imaging software (National Institute of Health, Maryland, USA).

### Effect of Superimposed AC

Superimposing an AC field to a DC, introduces three new parameters into the system—waveform type, AC peak-to-peak voltage and frequency. The effect of these parameters on the formation of microbubbles were investigated in these Examples to understand their effect on bubble diameter. The first series of experiments were conducted by testing two different applied AC voltages—2 kV_{P-P} and 4 kV_{P-P}. For 2 kV_{P-P}, the waveform oscillates 1 kV either side of the zero marker, and for 4 kV_{P-P}, the wave oscillates 2 kV from 0 to peak, and 0 to trough.

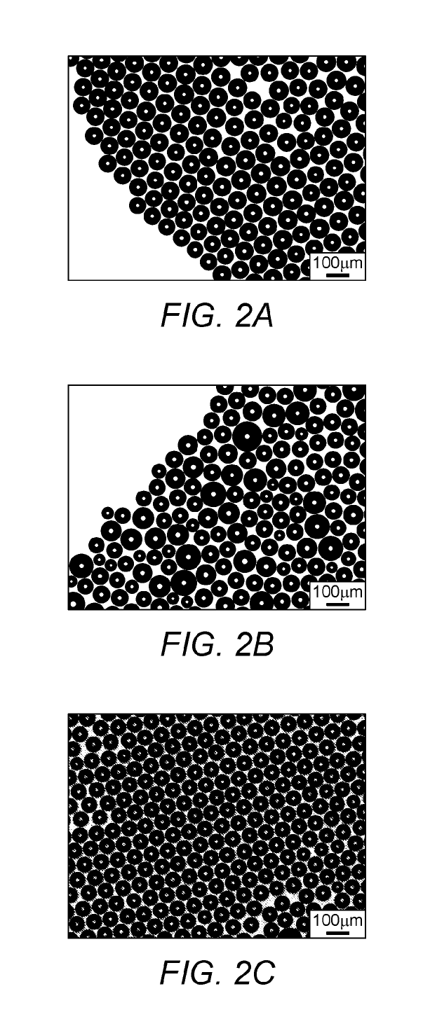

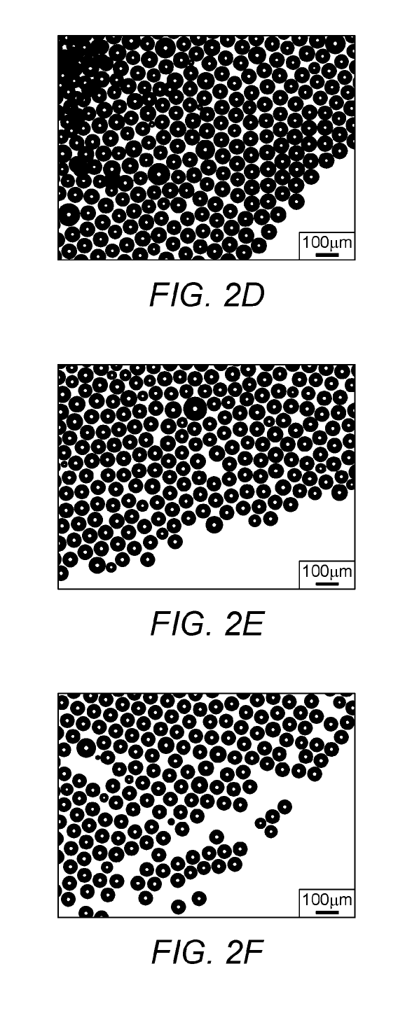

For these tests, the waveform used was a sinusoidal wave with a frequency of 500 Hz. The applied AC was modulated by varying the applied Peak-to-Peak voltage on the waveform generator. Once the _{P-P} AC was set, the DC voltage was increased from 2 kV up till 10 kV by increasing the DC offset. FIGS. 2(a) to 2(f) shows optical micrographs of microbubbles obtained by progressively increasing the applied DC voltage. It can be seen that increasing the DC voltage at this superimposed AC voltage causes a drop in bubble diameter. The average bubble diameter without an application of an electric field was ˜110 μm, when a DC voltage of 2 kV was applied, the average bubble diameter dropped to ˜80 μm. Increasing the applied DC voltage to 10 kV further reduces the bubble diameter to ˜55 μm, however increasing the voltage past 10 kV increased the polydispersity of the bubbles due to ionization of the surrounding air.

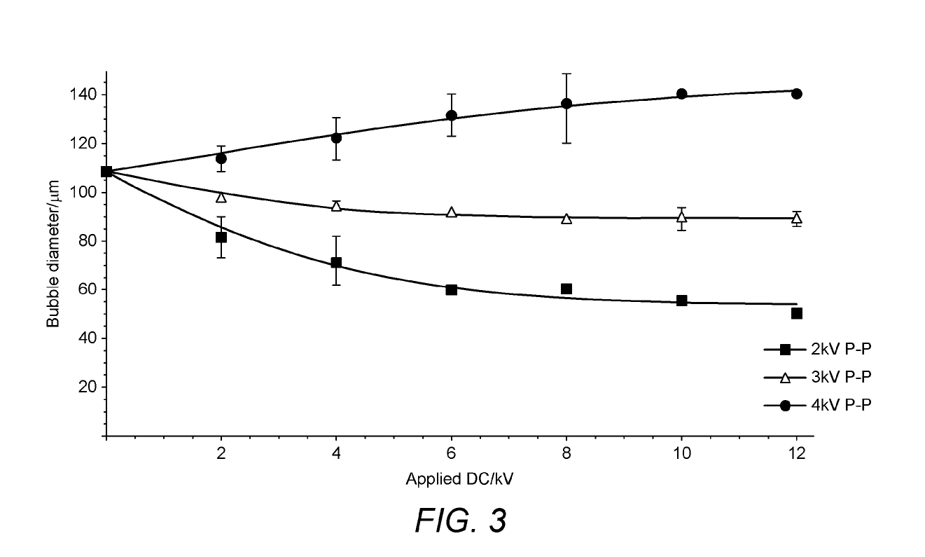

On the other hand, increasing the AC voltage to 4 kV (i.e. for an applied DC voltage of 8 kV, the waveform will oscillate between 6 kV and 10 kV), had the opposite effect on the bubble diameter. This is shown in FIG. 3.

This could be due to the fact that prolonged exposure of the bubble to high AC fields combined with high frequency, can result in the superheating of the bubble. Bubbles may tend to grow under ambient conditions as a result of evaporation at the gas-liquid interface.

Heat generated by high AC fields may have also contributed to heating effects which increase the overall bubble growth time. The effect of superimposed AC is summarised in FIG. 3 indicating that a superimposed AC voltage of 2 kV_{P-P} is optimal for this work.

### Effect of Frequency

Microbubbles were then obtained at different frequencies, at a constant superimposed AC voltage. Frequencies of 100 Hz, 500 Hz and 600 Hz were tested. Based on the results acquired for the effect of superimposed AC voltage on bubble diameter, an optimal AC voltage of 2 kV_{P-P} was maintained for all the tests. When a low frequency of 100 Hz was utilized, the bubble diameter dropped from ˜110 μm to ˜100 μm as the applied DC voltage was increased in steps of 2 kV up till 12 kV.

Increasing the applied frequency to 500 Hz resulted in a drop in bubble diameter from ˜110 μm to ˜55 μm when the applied DC voltage was increased from 0-12 kV in steps of 2 kV. Optical micrographs acquired shows that the largest drop in bubble diameter was observed at a low DC voltage of 2 kV, however, it was observed that a wide size distribution was prevalent. Microbubbles acquired at 6 kV displayed near-perfect monodispersity. An application of 12 kV was found to have a negative effect on the monodispersity and stability of the microbubbles.

Increasing the frequency to 600 Hz displayed interesting results, at an applied DC voltage of 8 kV, the bubble size was recorded to be on an average 38 μm, but increasing the voltage further past this point resulted in a foam-like cluster. This coalescence maybe due to the effects of heating due to presence of high electric fields.

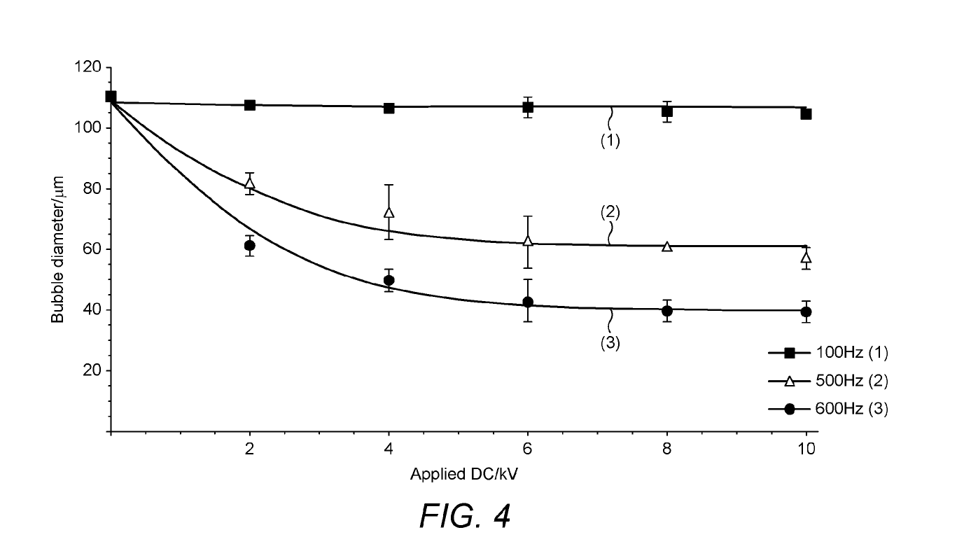

From the results presented above, it can be seen that frequency is an important parameter in these studies. As the frequency is increased, there is a clear and significant decrease in microbubble diameter (as illustrated in FIG. 4).

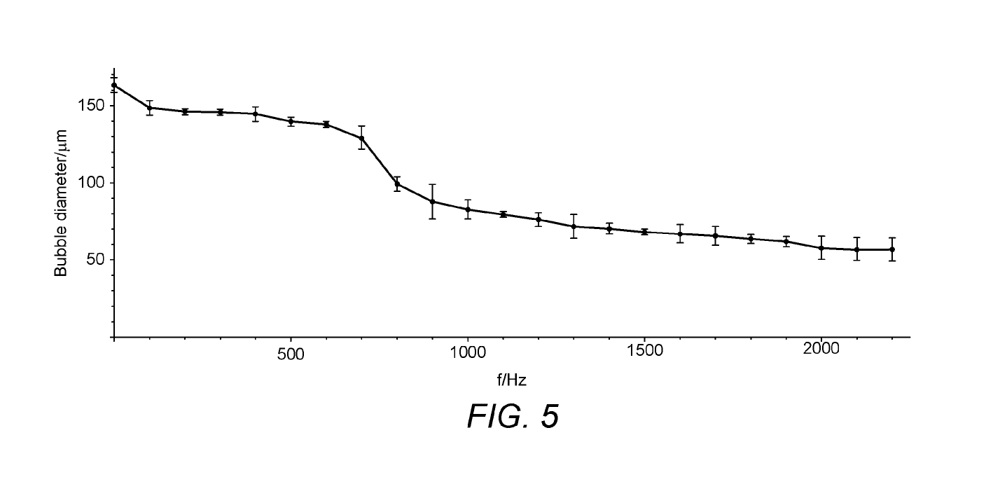

The experimental results (see FIG. 5) indicated a significant drop in bubble diameter between 100 Hz-2200 Hz. The applied DC voltage causes the elongation of the fluid stream, and the AC excitation facilitates droplet break-up. In order to reduce the bubble to 2 μm, a frequency of 10 MHz ought to be applied.

1. A process for producing microbubbles, the process comprising
passing a first fluid medium through a first capillary,
passing second fluid medium through a second capillary,
such that the first fluid medium and second fluid medium meet at a junction and form microbubbles having a core of the first fluid medium and a shell of the second fluid medium, the microbubbles then flowing along an exit capillary,
wherein a DC voltage and a superimposed AC voltage are applied to the exit capillary and microbubbles then exit the exit capillary.

2. A process according to claim 1, wherein a peak-to-peak AC voltage of 3 kV or less is applied to the exit capillary.

3. A process according to claim 1, wherein a peak-to-peak AC voltage of from 1.5 to 2.5 kV is applied to the exit capillary.

4. A process according to any one of the preceding claims, wherein the DC voltage applied to the exit capillary is from 1 to 12 kV.

5. A process according to any one of the preceding claims, wherein the DC voltage applied to the exit capillary is from 4 to 8 kV.

6. A process according to any one of the preceding claims, wherein the frequency of the superimposed AC voltage applied to the exit capillary is at least 500 Hz.

7. A process according to any one of the preceding claims, wherein the frequency of the superimposed AC voltage applied to the exit capillary is at least 800 Hz.

8. A process according to any one of the preceding claims, wherein the frequency of the superimposed AC voltage applied to the exit capillary is at least 2000 Hz.

9. A process according to any one of the preceding claims, wherein the frequency of the superimposed AC voltage applied to the exit capillary is at least 10,000 Hz.

10. A process according to any one of the preceding claims, wherein the frequency of the superimposed AC voltage applied to the exit capillary is at least 1 MHz.

11. A process according to any one of the preceding claims, wherein the first, second and exit capillaries each have an internal diameter of from 10 μm to 200 μm.

12. A process according to any one of the preceding claims, wherein the first, second and exit capillaries each have an internal diameter of from 50 μm to 150 μm.

13. A process according to any one of the preceding claims, wherein the first, second and exit capillaries are arranged in a T-junction, such that the first capillary is aligned with the exit capillary, and the second capillary is aligned substantially perpendicular to the first capillary and exit capillary.

14. A device for producing microbubbles comprising
a first capillary,
a second capillary,
a junction, at which the first capillary and second capillary meet, and an exit capillary extending away from the junction, and
a power supply connected to the exit-capillary, the power supply adapted to apply a DC voltage and a superimposed AC voltage to the exit capillary.

15. The device according to claim 14, wherein the power supply is adapted to supply a peak-to-peak AC voltage of 3 kV or less to the exit capillary.

16. The device according to claim 14 or 15, wherein the power supply is adapted to supply a peak-to-peak AC voltage of from 1.5 to 2.5 kV to the exit capillary

17. The device according to any one of claims 14 to 16, wherein the power supply is adapted to apply a DC voltage to the exit capillary of from 4 to 8 kV.

18. The device according to any one of claims 14 to 17, wherein the power supply is adapted to apply an AC voltage to the exit capillary with a frequency of at least 500 Hz.

19. The device according to any one of claims 14 to 18, wherein the power supply is adapted to apply an AC voltage to the exit capillary with a frequency of at least 800 Hz.

20. The device according to any one of claims 14 to 19, wherein the power supply is adapted to apply an AC voltage to the exit capillary with a frequency of at least 2000 Hz.

21. The device according to any one of claims 14 to 20, wherein the power supply is adapted to apply an AC voltage to the exit capillary with a frequency of at least 10,000 Hz.

22. The device according to any one of claims 14 to 21, wherein the power supply is adapted to apply an AC voltage to the exit capillary with a frequency of at least 1 MHz.
